(19) United States
(12) Patent Application Publication
(10) Pub. No.: US 2025/0090302 A1
(43) Pub. Date: Mar. 20, 2025

(54) CORRECTING THE REFRACTION OF AN EYE BY CORNEAL MODIFICATION

(71) Applicant: Carl Zeiss Meditec AG, Jena (DE)

(72) Inventors: Mark BISCHOFF, Jena (DE); Johannes KINDT, Weimar (DE)

(21) Appl. No.: 18/691,816

(22) PCT Filed: Sep. 15, 2022
(86) PCT No.: PCT/EP2022/075612

§ 371 Date: Nov. 4, 2024

(30) Foreign Application Priority Data
Sep. 17, 2021 (DE) 10 2021 124 087.5

(51) Int. Cl. A61F 2/14

(52) U.S. Cl. CPC A61F 2/1451; A61F 2250/0036; A61F 2250/0053

(57) ABSTRACT

The invention relates to a method for producing control data for correcting the refraction of an eye (2) by corneal modification, comprising the steps of receiving data about a refraction correction need for the eye (2), determining, on the basis of the refraction correction need, an additive refraction alteration value by an implant (26) to be inserted into the cornea (17), determining a subtractive refraction alteration value by a lenticule (21) to be removed from the cornea (17), the additive refraction alteration value and the subtractive refraction alteration value together yielding the refraction correction need, calculating a lenticule (21) to be isolated in the cornea (17), the calculation being implemented in such a way on the basis of the subtractive refraction alteration value that the lenticule (21) is embodied to bring about the subtractive refraction alteration value as a result of being removed from the cornea (17).

The invention relates to a method for correcting the refraction of an eye by means of corneal modification, and to a corresponding apparatus.

Attachment lenses in the form of spectacles have been used since time immemorial to correct human refractive errors. Recent times have seen various approaches of correcting the refractive error of the eye by virtue of modifying the cornea. The modification is intended to ensure a change in the curvature of the cornea. The corneal front surface must be flattened for the purpose of correcting myopia, which is why the volume to be removed is thicker in the middle, which is to say in the region of the visual axis, than at the edge. By contrast, the front surface of the cornea must be curved more strongly in order to correct hyperopia, which is why the volume to be removed is thicker at the edge than in the middle. As a result, the overall imaging properties of the eye are influenced so that a refractive error is reduced or, in the ideal case, even entirely compensated for.

A very successful method in this respect was developed by Carl Zeiss Meditec AG and called SMILE. It uses pulsed laser radiation to isolate a lenticule in the cornea, which can then be removed from the cornea through a lateral opening cut which leads to the corneal surface of the eye and serves as a work channel. The volume of the lenticule is structured and dimensioned such that the front surface of the cornea changes its curvature as required for the correction. The method brings about subtractive correction since volume is removed.

Another approach introduces implants into the cornea of the eye. To this end, a slot is introduced into the cornea and the implant is inserted therein. It is designed so that it changes the curvature of the front surface of the cornea as desired. This approach consequently brings about an additive correction since volume is added. WO 2016/050711 A1 rectifies one problem of this additive correction, which consists of the fact that stresses occur in the cornea of the eye at the edge of the implant. To solve this, the integration of the implant into the cornea of the eye is additionally assisted by relief cuts in the cornea of the eye.

According to U.S. Pat. No. 5,722,971 A, a chamber referred to as a “pocket” is created, into which a solid or partly solid material can be inserted. Three variants are mentioned in this respect. In a first variant, the solid or partly solid material overfills the “pocket”; this is additive correction. In a second variant, the solid or partly solid material underfills the “pocket”; this results in a subtractive correction since less material is present in the cornea post procedure than before. In a third variant, the solid or partly solid material precisely fills this “pocket”. This change is volume neutral and a modification of the optical power of the cornea can only be achieved thereby if the solid or partly solid material has a different refractive index to that of the cornea. The document provides for the eye to be measured after the creation of the chamber, in order then to define which implant should be inserted.

The introduction of material into the cornea of the eye is also found in WO 2008/131888 A1, there for transplantation purposes, which is to say for the replacement of a damaged, for example opaque, cornea of the eye. The part of the cornea of the eye to be replaced is bounded by laser radiation and removed through an opening cut likewise created by laser radiation. Donor material is introduced into the chamber created thus, with the result that the damaged part of the cornea of the eye is replaced. This does not yet achieve additive correction per se if the donor material replaces the existing cornea one to one. However, the aforementioned publication also mentions that the donor material to be inserted can be slightly larger or smaller than the chamber in terms of its dimensions in order to correct an existing refractive error at the same time, which is to say in order to advantageously influence the curvature of the corneal front side. Additive correction is brought about if the donor material to be inserted is slightly larger in terms of its dimensions, and it is subtractive correction if said donor material is slightly smaller.

DE 102013218415 A1 considers the integration of a presbyopia implant, which is provided for the correction of presbyopia. Its purpose is that of designing the passage of the radiation through the cornea of the eye differently in regions close to the visual axis than in regions away from the visual axis, and it should increase the depth of field. DE 102013218415 A1 provides cylindrical projections in particular, which are formed to secure the presbyopia implant against slippage in the cornea of the eye. In one example, this presbyopia implant is in the form of a ring-like disk which improves the depth of field of the optical imaging, and has an advantageous influence on the presbyopia. In this respect, a lenticule extraction method, like in SMILE, is combined in this document with an implantation method in which an implant is placed in the cornea following the removal of the lenticule. However, a refraction correction effect by the implant is not discussed any further in DE 102013218415 A1; rather, there is a discussion to the effect that a corrective effect going beyond the presbyopia arises as a result of the extraction of a lenticule. Thus, this document envisages a subtractive approach for refraction correction.

The problem addressed by the invention is that of specifying a method for establishing control data for correcting the refraction of an eye by corneal modification and specifying a corresponding apparatus which rectifies the disadvantages in the prior art and in particular ensures a refractive error correction which is as reliable as possible over a wide diopter range, especially in the case of hyperopia.

The invention is characterized in the independent claims. The dependent claims relate to preferred developments.

The invention combines the insertion of an implant into the cornea of the eye—an additive correction method—with the removal of a lenticule-a subtractive correction method. In the process, an implant intended to be inserted into the cornea is initially determined in respect of the additive refraction alteration value caused thereby using data about a refraction correction need for the eye as a starting point. This additive refraction alteration value is clearly defined, for example by the dimensions and spatial design of the implant, and easy to calculate or known for an implant. However, it does not correct the refractive error completely in any embodiment. The three variants explained below relate to aspects related to this only incomplete correction achieved by the additive refraction alteration value. A subtractive refraction alteration value is calculated once the implant is fixed, at least in respect of its additive refraction alteration value. It corresponds to the difference between the refraction correction need required overall and the additive refraction alteration value determined earlier. Subsequently, the subtractive refraction alteration value is used to calculate a lenticule to be isolated in the cornea, in such a way that said lenticule brings about the subtractive refraction alteration value as a result of being removed from the cornea or following its (separate) removal therefrom. Finally, a cut surface which isolates the lenticule in the cornea is calculated, and hence a subsequent removal is prepared. This isolating cut at the same time bounds a chamber for the implant to be inserted, the chamber being empty once the lenticule has been removed.

Thus, within the scope of a combination of additive refractive error correction (insertion of a refraction-altering implant) and subtractive refractive error correction (removal of a refraction-altering lenticule), the invention for the first time proceeds from the refraction alteration caused by the implant once inserted in the cornea. A subtractive refraction correction to be provided by the lenticule is calculated on the basis of this refraction alteration. The prior art has not followed this approach since, with regards to the alteration of the refraction therein, work was carried out exclusively with additive or subtractive refractive error correction (implant according to WO 2016/050711 A1; lenticule removal according to DE 102013218415 A1), the interaction between additive and subtractive refractive error correction was not analyzed in any more detail (U.S. Pat. No. 5,722,971 A), or the removal of the lenticule was not intended to bring about or be connected with any refractive change at all (transplantation approach of WO 2008/131888 A1).

Combining additive and subtractive refraction influences and starting from the additive refraction alteration value or implant have particular and surprising advantages. It is no longer problematic that, following the removal or taking of a lenticule which might be very thick at the edge or at the center in the case of significant corrections, an exclusively subtractive correction might lead to a minimum thickness critical for sufficient stability being undershot. This restriction linked to subtractive methods comes to bear when correcting both hyperopia and myopia but is particularly problematic when correcting hyperopia since the maximum thickness of the lenticule to be removed is located at the edge of the lenticule in that case, and this is physiologically disadvantageous. The problem of a type of step following the removal of the lenticule arises in that case. This can be avoided by the combination of additive and subtractive refractive error corrections.

A further advantage arises in a first variant if the additive refraction alteration value or implant is determined in such a way that it brings about an overcorrection of the hyperopia or myopia. In this context, overcorrection is understood to mean that an existent hyperopia or myopia is not only compensated for but (if use were made of the implant only) converted into the opposite, specifically a myopia or hyperopia. In this respect, the correction is too strong. However, this result does not occur since the subtractive residual refraction correction comprises a corresponding reversed myopia or hyperopia correction. At first glance, it appears counterproductive to implement any type of correction, be it additive or subtractive, as an overcorrection, which is to say introduce or remove more material than would actually be necessary for the correction. However, the overcorrection by the additive refraction alteration value or implant is linked to the great advantage that the basic geometry of the implant is similar to that of the lenticule to be removed. In the case of hyperopia, the implant to be inserted has its maximum thickness in the middle and is thinner at the edge, which is to say in regions away from the axis, since the corneal front surface should be curved more strongly. On account of the overcorrection, the implant itself would bring about an increase in curvature that is too great. This is compensated for by the subtractive refraction alteration value or lenticule, which therefore likewise has a volume that is thicker in the middle than at the edge. Hence, in a sectional view, the implant and the lenticule have the same basic shape with a greater thickness at the center and a lesser thickness at the edge. The chamber provided by the lenticule removal then has a basic shape that is ideal for accommodating the implant. An analogous statement applies in the inverse case of a myopia correction, in which both the implant and, on account of the required overcorrection compensation, the lenticule are thinner at the center than at the edge.

This procedure appearing counterproductive at first glance consequently facilitates the introduction of the implant since the chamber provided by the lenticule has the same basic shape as the implant. It is self-evident that the individual dimensions of lenticule and implant are not identical; however, the correspondence in the basic shape facilitates the insertion significantly and in particular avoids stresses at the edge of the chamber, as are the subject matter of WO 2016/050711 A1.

This particularly applies if the volume of the implant is no greater than that of the lenticule. This can easily be set by way of a suitable choice of the two refraction alteration values.

A second variant is advantageous in that a rotational position of the implant no longer presents any problems, even in the case of a rotationally asymmetric, in particular astigmatic correction. When inserting the implant, non-rotationally symmetric in that case, conventional approaches must ensure that the rotational position of said implant matches the orientation (axis) of the astigmatism. Further, there had to be precautions in place to ensure that the implant does not undesirably rotate out of this predetermined position over time. The choice of the two refraction alteration values, which is to say the refraction alteration by the implant and the refraction alteration by the lenticule, now allows the provision of a purely rotationally symmetric implant which has the same refraction alteration as a consequence, independently of its rotational position in the cornea of the eye. The rotational asymmetry required overall is subsequently brought about exclusively by the subsequently established lenticule, which is to say the subtractive refraction alteration value. Since the lenticule is isolated and ultimately defined by a cut surface in the cornea of the eye, this rotational asymmetry can be set very easily. For example, astigmatic corrections are well known for the SMILE method. The chamber into which the rotationally symmetric implant is inserted is rotationally asymmetric, but this cannot change over time even if the implant should twist within the chamber. The starting point according to the invention of the refraction alteration achieved by the implant therefore enables very simple control even of rotationally asymmetric corrections without special precautions needing to be made with regards to anchoring the implant against rotating positional changes in the chamber.

The implant to be inserted is preferably multifocal, for example to bring about a presbyopia correction. Presbyopia is understood to mean age-related hyperopia caused by the loss of accommodation. For example, a presbyopia-correcting multifocal implant is known from WO 2021/156203 A1. The latter comprises a lens body having two concentric zones which comprise different diffraction structures from one another. The diffraction structures are designed such that they provide a plurality of foci for specific wavelengths in the range of visible light.

Until now, the use of implants always proceeded on the assumption that the implants must essentially be fabricated on an individual basis for patients in order to achieve a complete correction or a desired corneal modification in full. The starting point of the refraction alteration of the implant and the adaptation of the lenticule to the remaining residual refraction correction now allows work to be carried out with standardized implants in a further variant. In this respect, it is envisaged that a set of implants is provided, which are each designed for refraction alteration by way of an insertion into the cornea, the set comprising a plurality of implants each with an individual additive refraction alteration value. Then, determining the additive refraction alteration value comprises the selection of one of these implants from the set. This approach allows industrial production of implants. In this case, care no longer needs to be taken that the material used for the implants allows retrospective patient-individual processing (in the laboratory or in situ). It is possible to use materials which are very much better matched to biocompatibility. Advantageously, the implant whose refraction alteration is closest to the refraction correction need will be selected when making a selection from the set. It is self-evident that the property that the implant brings about an overcorrection in this case and/or has a purely rotationally symmetric design can be also used here, for example by way of a set made up of spherical implants.

The described method for creating control data comprises the preparation for correcting the refraction of an eye by corneal modification and requires no surgical step. However, it may be complemented by the latter to form a surgical method. In this step, a laser apparatus is used to create the cut surface, the lenticule is removed from the cornea, and the implant is inserted into the chamber in the cornea remaining after the removal of the lenticule. In this case and very much as a matter of principle, the method can be carried out by a computer, in particular comprising a processor. This computer can be designed as a planning station, as known from elsewhere in the prior art.

In this respect, the invention further includes a software product for carrying out the aforementioned method since the taking account of the refraction correction need, the determination of the implant, and the calculation of the residual refraction correction and the cut surface can be implemented without problems by appropriate software.

The apparatus provided for solving the problem corresponds in terms of its approach to the method explained, wherein provision is made for a calculation device which is configured to carry out the corresponding method steps.

To the extent that reference is made above to spherical and astigmatic corrections, these should be understood to be merely examples of rotationally symmetric refraction alterations and non-rotationally symmetric refraction alterations. Higher-order corrections can be carried out in this way by all means. Further, with regards to the case of overcorrection, the latter is at least related to a major axis of the rotational asymmetry in the case of a non-rotationally symmetric refraction correction need; specifically to the axis with the smaller curvature error in the case of astigmatism. Thus, for example, there may be overcorrection for this major axis but no overcorrection for the axis lying at 90° thereto. Ideally, the correction need here is exactly covered along one axis by the implant. Should this not be possible, the overcorrection should preferably be present for both axes in order to ensure the desired correspondence in the basic structure between implant and lenticule.

To the extent that reference is made here to the determination of the implant, this relates to defining the additive refraction alteration value, for example comprising the geometric data of the implant, which is to say for example its extents. A complete geometric description of the volume adopted by the implant is possible in a development. Should a set of (standard) implants be provided, the determination of the additive refraction alteration value may also relate to the selection of an implant from the set since the respective additive refraction alteration value is known for the individual implants in the set. Ultimately, the subsequent processes re-quire the refraction alteration of the implant to be defined. In this case, determining the implant and calculating the residual refraction correction can also be combined by virtue of the refraction correction need being broken down into a refraction alteration brought about by the implant and a residual remainder. Then, this remainder is the residual refraction correction. In this way, the refraction alteration has been defined first as essential parameter for the implant and this has been used to determine the implant in a very compact procedure, and the residual refraction correction, for which the lenticule and the cut surface bounding the lenticule are subsequently suitably established, has also been calculated at the same time.

It is necessary to distinguish between the correction respectively brought about by the extraction of the lenticule and the insertion of the implant and the refractive power of the lenticule or implant itself.

It goes without saying that the features mentioned above and the features yet to be explained hereinafter can be used not only in the specified combinations but also in other combinations or on their own, without departing from the scope of the present invention.

The invention will be explained in even greater detail below on the basis of exemplary embodiments with reference to the accompanying drawings, which likewise disclose features essential to the invention. These exemplary embodiments are provided for illustration only and should not be construed as limiting. For example, a description of an exemplary embodiment having a multiplicity of elements or components should not be construed as meaning that all of these elements or components are necessary for implementation. Rather, other exemplary embodiments may also contain alternative elements and components, fewer elements or components, or additional elements or components. Elements or components of different exemplary embodiments can be combined with one another, unless indicated otherwise. Modifications and variations that are described for one of the exemplary embodiments can also be applicable to other exemplary embodiments. In order to avoid repetition, elements that are the same or correspond to one another in different figures are denoted by the same reference signs and are not explained repeatedly. In the figures:

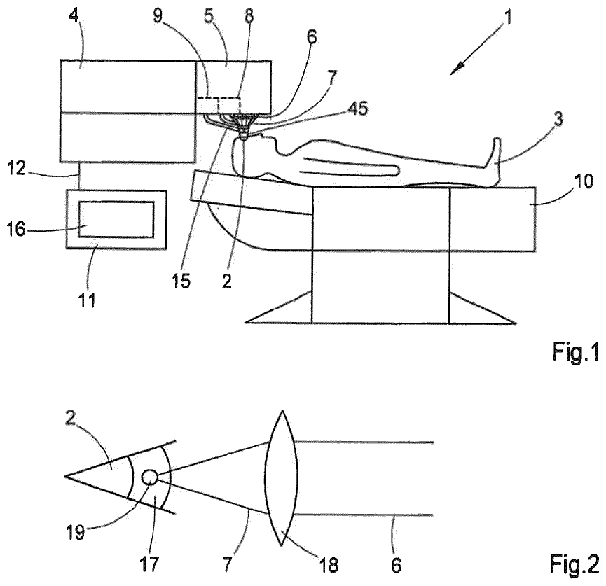

FIG. 1 shows a schematic illustration of a treatment apparatus having a planning device for ophthalmosurgical refraction correction,

FIG. 2 shows a schematic illustration of the effect of the laser radiation which is used in the treatment apparatus of FIG. 1,

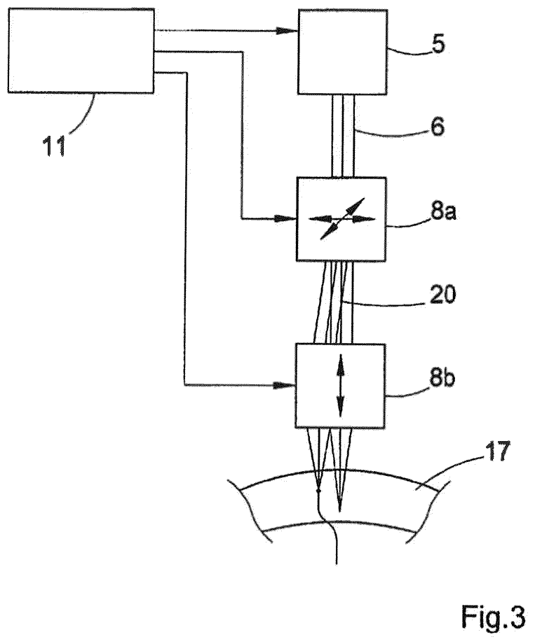

FIG. 3 shows a further schematic illustration of the treatment equipment of FIG. 1, in relation to the introduction of laser radiation,

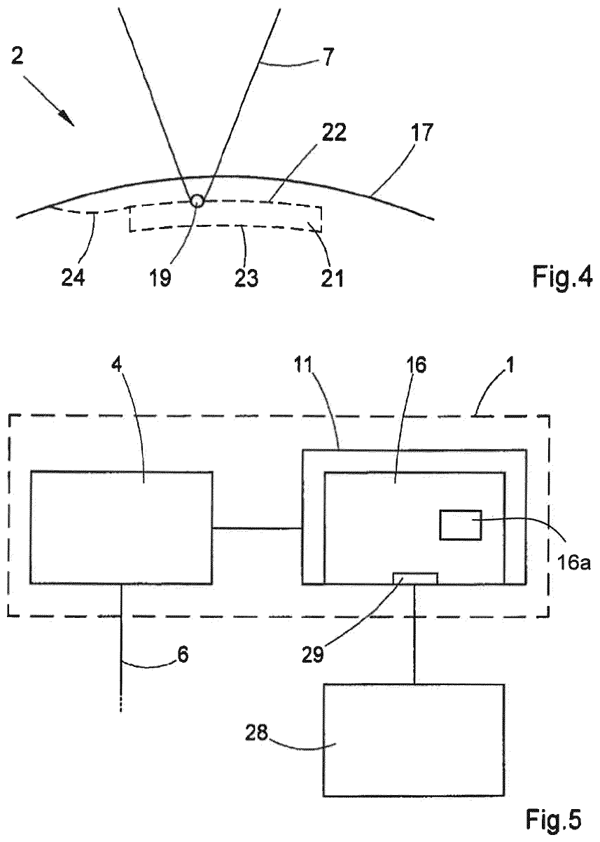

FIG. 4 shows a schematic sectional illustration through the cornea of the eye for elucidating the removal of the corneal volume in the context of the ophthalmosurgical refraction correction,

FIG. 5 shows a schematic illustration in respect of the design of the treatment equipment of FIG. 1 with particular reference to the planning device present there,

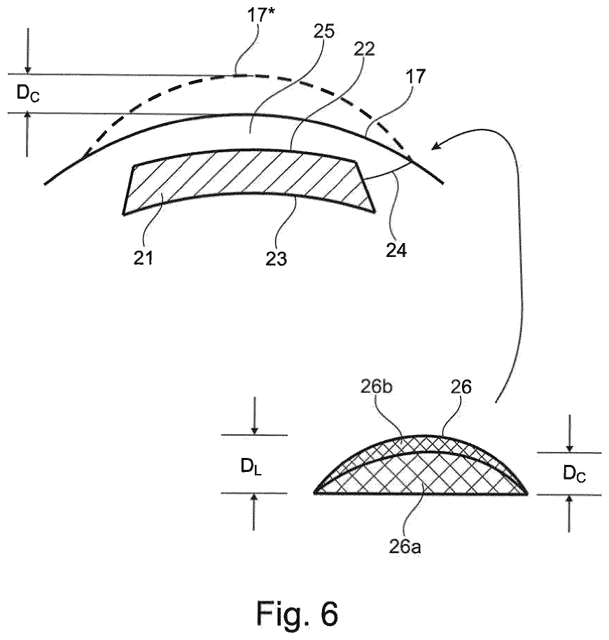

FIG. 6 shows a schematic illustration for elucidating a synergy of additive and subtractive refractive error correction,

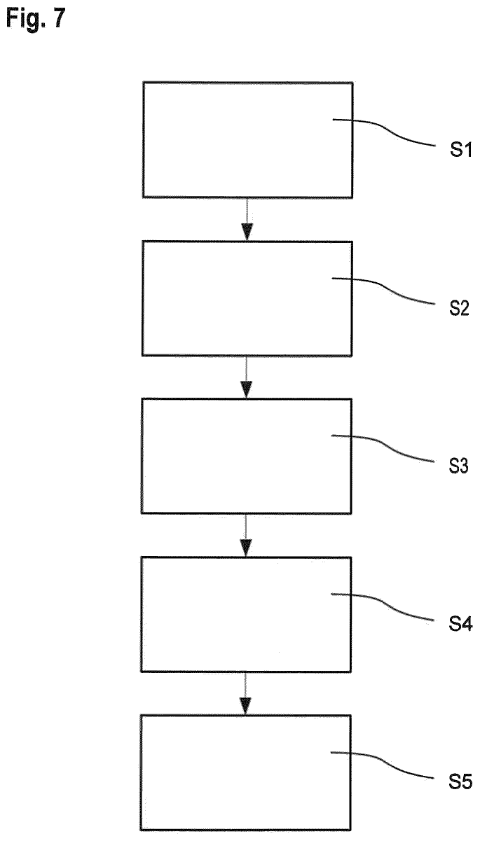

FIG. 7 shows a block diagram for a method for establishing essential data for the surgical method, for example of FIG. 6,

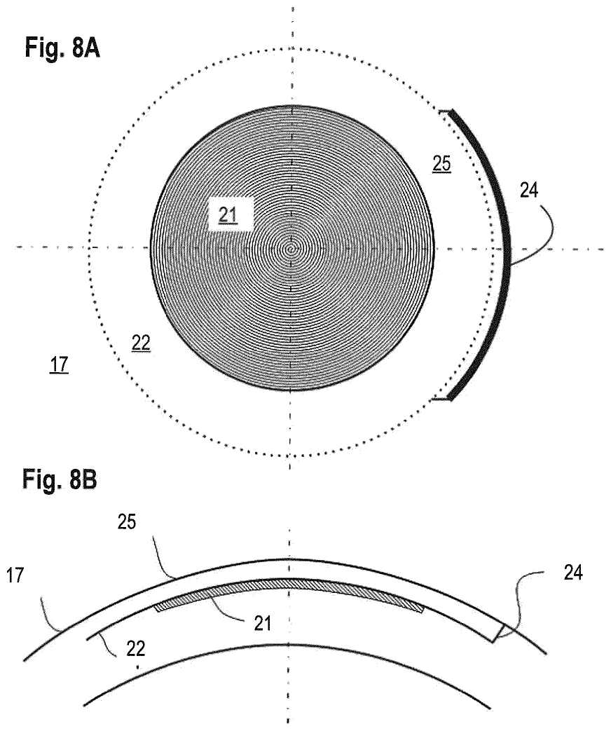

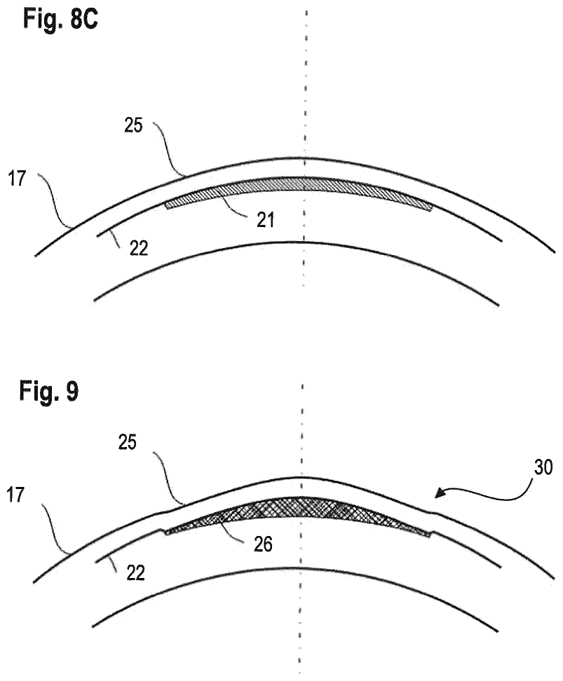

FIGS. 8A to 8C show different views of the cornea of an eye for creating a cut surface for removing a lenticule, wherein FIG. 8A shows a plan view of the cornea and FIG. 8B reproduces a sectional illustration along a vertical axis in FIG. 8A, and FIG. 8C reproduces a sectional illustration along a horizontal axis in FIG. 8A, and

FIG. 9 shows a sectional illustration similar to that in FIG. 8C after the insertion of an implant.

A treatment apparatus 1 for ophthalmic surgery is depicted in FIG. 1. The treatment apparatus 1 is designed to introduce laser cuts on an eye 2 of a patient 3. To this end, the treatment apparatus 1 comprises a laser device 4 which emits a laser beam 6 from a laser source 5, the laser beam being directed into the eye 2 or the cornea of the eye as a focused beam 7. Preferably, the laser beam 6 is a pulsed laser beam with a wavelength between 300 nanometers and 10 micrometers. Further, the pulse length of the laser beam 6 is in the range between 1 femtosecond and 100 nanoseconds, with pulse repetition rates of 50 to 20000 kilohertz and pulse energies between 0.01 microjoules and 0.01 millijoules being possible. The treatment apparatus 1 creates a cut surface in the cornea of the eye 2 by deflecting the pulsed laser radiation. To this end, a scanner 8 and a radiation intensity modulator 9 are provided in the laser device 4 or the laser source 5 thereof.

The patient 3 is situated on for example a couch 10, which is adjustable in three spatial directions in order to suitably align the eye 2 in relation to the incidence of the laser beam 6. In a preferable construction, the couch 10 is adjustable in motor-driven fashion. It is alternatively possible to adjust the laser device 4. In particular, the control can be implemented by a controller 11 which, in principle, controls the operation of the treatment apparatus 1 and, to this end, is connected to the treatment apparatus by way of suitable data links, for example connection lines 12. Naturally, this communication can also be implemented in different ways, for example via light guides or by radio. The controller 11 makes appropriate adjustments to and controls the timing of the treatment apparatus 1, in particular the laser device 4, and hence brings about an appropriate method sequence on the treatment apparatus 1.

The treatment apparatus 1 comprises a fixation device 15, which fixates the relative position of the cornea of the eye 2 with respect to the laser device 4. This fixation device 15 may comprise a known contact glass 45, to which the cornea of the eye is applied by negative pressure and which imparts a desired geometric shape on the cornea of the eye. Such contact glasses are known to a person skilled in the art from the prior art, for example from DE 102005040338 A1. The disclosure of this document, to the extent this relates to the description of the structure of the contact glass 45 that is suitable for the treatment apparatus 1, is completely incorporated herein.

The treatment device 1 further comprises a camera (not illustrated here), which is able to record an image of the cornea 17 of the eye through the contact glass 45. In this case, the lighting for the camera may be implemented both in the visible and in the infrared spectral range.

The controller 11 of the treatment apparatus 1 comprises a planning device 16 which will still be explained in detail below and which comprises at least one calculation device which, for preparation purposes, calculates the cut surface(s) and/or control data, in particular actuation data for the treatment apparatus, so that the cut surface(s) can be created within the scope of the surgical method.

FIG. 2 schematically shows the effect of the incident laser beam 6. The laser beam 6 is incident into the cornea 17 of the eye 2 as a focused laser beam 7. Schematically plotted optics 18 are provided for focusing purposes. They bring about a focus 19 in the cornea 17, the laser radiation energy density being so high in said focus that, in combination with the pulse length of the pulsed laser radiation 6, a nonlinear effect occurs in the cornea 17. By way of example, each pulse of the pulsed laser radiation 6 can create an optical breakdown in the cornea 17 of the eye in the focus 19, said breakdown, in turn, initiating a plasma bubble as indicated merely schematically in FIG. 2. When the plasma bubble arises, the tissue layer separation comprises an area larger than the focus 19, even though the conditions for creating the optical breakdown are only obtained in the focus 19. So that an optical breakdown is created by each laser pulse, the energy density, which is to say the fluence of the laser radiation, must lie above a certain, pulse-length-dependent threshold value. A person skilled in the art is aware of this relationship, for example from DE 69500997 T2. Alternatively, a tissue-separating effect can also be achieved by the pulsed laser radiation by virtue of a plurality of laser radiation pulses being emitted into a region, with the focal spots overlapping. Then, a plurality of laser radiation pulses interact in order to obtain a tissue-separating effect. The type of tissue separation used for the treatment apparatus 1 is of no further relevance to the description below, however; all that is essential is that a cut surface is produced in the cornea 17 of the eye 2.

In order to correct the refraction by ophthalmic surgery, a corneal volume referred to as lenticule is removed from a region within the cornea 17 by means of the laser radiation 6 by virtue of tissue layers being separated within the cornea, said tissue layers isolating the corneal volume and then enabling the removal thereof. The corneal volume is bounded by a three-dimensionally shaped cut surface. To this end, the position of the focus 17 of the focused laser radiation 7 in the cornea 17 is adjusted in three dimensions in the case of the laser radiation introduced in pulsed form. This is shown schematically in FIG. 3. The curvature of the front side of the cornea 17 is modified in a targeted fashion by the removal of the lenticule in order thus to attain the correction of the refraction. A uniformly thick volume would not substantially change the curvature of the front side of the cornea-hence the term lenticule is used.

FIG. 3 only plots elements of the treatment apparatus 1 to the extent that these are necessary for understanding the cut surface production. As already mentioned, the laser beam 6 is focused at a focus 19 in the cornea 19 and the position of the focus 19 in the cornea is adjusted such that, for the purposes of producing cut surfaces, energy of laser radiation pulses, focused at different positions, is introduced into the tissue of the cornea 17. The laser radiation 6 is provided, preferably as pulsed radiation, by the laser source 5. In the structure of FIG. 3, the scanner 8 has a two-part structure and consists of an xy-scanner 8a, which is realized in one variant by two galvanometer mirrors that deflect substantially orthogonally. The scanner 8a two-dimensionally deflects the laser beam 6 coming from the laser source 5 such that a deflected laser beam 20 is present downstream of the scanner 9. Consequently, the scanner 8a brings about an adjustment of the position of the focus 19 substantially perpendicular to the principal direction of incidence of the laser beam 6 in the cornea 17. In addition to the xy-scanner 8a, a z-scanner 8b is additionally provided in the scanner 8 for adjusting the depth position, said z-scanner being embodied as an adjustable telescope, for example. The z-scanner 8b ensures that the z-position of the position of the focus 19, which is to say the position thereof on the optical axis of incidence, is modified. The z-scanner 8b can be disposed upstream or downstream of the xy-scanner 8a. The scans displace for example the focus 19 along a three-dimensional trajectory, along which the laser pulses are emitted in order to form the cut surface(s).

For the principle of operation of the treatment apparatus 1, the assignment of the individual coordinates to the spatial directions is not essential, just as it is not essential that the scanner 8a deflects about mutually orthogonal axes. Rather, it is possible to use any scanner that is able to adjust the focus 19 in a plane not containing the axis of incidence of the optical radiation. It is also possible to use any non-Cartesian coordinate system for the purposes of deflecting or controlling the position of the focus 19. Examples include spherical coordinates or cylindrical coordinates. The position of the focus 19 is controlled by means of the scanners 8a, 8b which are controlled by the controller 11, the latter making appropriate adjustments to the laser source 5, the modulator 9 (which is not shown in FIG. 3) and the scanner 8. The controller 11 ensures a suitable operation of the laser source 5 and the three-dimensional focus adjustment, explained here in exemplary fashion, such that ultimately a cut surface is formed which isolates a certain corneal volume that is intended to be removed or taken for correcting the refraction. In this case, it operates according to actuation data specified therefor.

For example, the actuation data are specified as target points for repositioning the focus and/or as data for the aforementioned trajectory. As a rule, the actuation data are combined in an actuation data record. The latter specifies geometric specifications of the cut surface to be formed, for example the coordinates of the target points as a pattern. Then, in this embodiment, the actuation data record also contains specific manipulated variables for the focal position adjustment mechanism, for example for the scanner 8. The actuation data are based on control data specifying the cut surface(s) to be created, as will be explained below.

The production of the cut surface using the treatment apparatus 1 is shown in FIG. 4 in exemplary fashion. A corneal volume 21 in the cornea 17 is isolated by repositioning the focus 19, at which the focused beam 7 is focused. A multi-part cut surface is formed, which is why reference is also made to “cut surface(s)”. In exemplary fashion, it comprises an anterior flap cut surface 22 and a posterior lenticule cut surface 23 in this case. These terms should be considered purely exemplary here and are only intended to establish the relationship to the conventional LASIK or flex method, for which the treatment apparatus 1 is optionally likewise designed. All that is essential here is that the cut surfaces 22 and 23 and, optionally, peripheral edge cuts which are not labeled here and bring together the cut surfaces 22 and 23 at their edges, surround and isolate the corneal volume 21. The corneal volume 21 can be taken through an opening cut 24, as provided by the SMILE method according to DE 102007019813 A1. The disclosure of this document is incorporated here in its entirety.

FIG. 5 schematically shows the treatment apparatus 1, and the importance of the planning device 16 should be explained in more detail on the basis thereof. In this variant, the treatment apparatus 1 comprises at least two devices or modules. The laser device 4, already explained above, emits the laser beam 6 onto the eye 2. As already explained, the operation of the laser device 4 is implemented fully automatically by way of the controller 11; i.e., following an appropriate start signal, the laser device 4 starts the creation and deflection of the laser beam 6 and creates cut surfaces constructed as described above. The laser device 5 receives the control signals required for the operation from the controller 11, which was provided with appropriate control data at an earlier time. For example, this is implemented by means of the planning device 16, which is shown purely in exemplary fashion as a constituent part of the controller 11 in FIG. 5. Naturally, the planning device 16 can also have an independent embodiment and communicate with the control device 11 in wired or wireless fashion. All that is essential in that case is that an appropriate data transfer channel is provided between the planning device 16 and the controller 11.

As core element, the planning device 16 comprises a calculation device 16a which, as yet to be explained below, calculates the cut surface(s) intended to be created in the cornea 17 and establishes the data required for these cut surfaces. The planning device 16, or the calculation device 16a directly, creates the actuation data record therefrom, the latter being made available to the controller 11 for the purpose of carrying out the ophthalmosurgical refraction correction.

To calculate the cut surface(s), the calculation device 16a uses measurement data regarding the cornea of the eye. In the embodiment described here, these measurement data originate from a measuring device 28 which had measured the eye 2 of the patient 2 earlier. Naturally, the measuring device 28 can be designed in any suitable way and transmit the corresponding measurement data to the interface 29 of the planning device 16.

The planning device assists the operator of the treatment apparatus 1 when defining the cut surface for isolating the corneal volume 21. This can go as far as fully automatically defining the cut surfaces, which may for example be implemented by virtue of the calculation device 16a determining, from the measurement data, the corneal volume 21 to be removed, defining the boundary face(s) of said corneal volume as cut surface(s) and creating appropriate control data for the controller 11 therefrom. At the other end of the degree of automation, the planning device 16 may provide input options, at which a user enters the cut surfaces in the form of geometric parameters, etc. Intermediate levels provide suggestions for the cut surfaces that are generated automatically by the planning device 16 and then are modifiable by a user. In principle, all the concepts already explained above in the more general part of the description may be applied here in the calculation device 16a.

FIG. 6 shows the underlying principle of the refractive error correction, for which the calculation device 16a provides the essential data and which can be carried out by means of the treatment apparatus 1. In this case, FIG. 6 shows a sectional illustration similar to that in FIG. 4, and corresponding elements have been denoted by the same reference signs as in FIG. 4. The up-per part of FIG. 6 shows the cornea 17 of the eye, with dashed lines being used to plot the cornea with an altered form, which sets in following the surgical procedure and which is denoted by 17*. It is evident that the front side of the cornea 17* has greater curvature following the surgical procedure. Here, in the drawing which is not to scale, an increase in curvature in the middle has been plotted with a dimension Dc. The lenticule 21 is isolated in the cornea by means of the aforementioned cut surfaces 22, 23, which together form a three-dimensional cut surface. Attention is drawn to the fact that, in the depicted embodiment, this lenticule 21 would not bring about the increase in curvature but would in fact flatten the curvature. This will be discussed below. Once the lenticule 21 has been isolated and removed through the opening cut 24, an implant 26, depicted schematically bottom right in FIG. 6, is incorporated. The incorporation procedure is symbolized by an arrow, which is intended to indicate that the lenticule 26 is introduced into the chamber through the opening cut 24, said chamber having been formed following the removal of the lenticule 21 and being bounded by the surfaces 22, 23. FIG. 6 shows that the lenticule 21 has a height DL that is greater than the height Dc, by which the curvature of the cornea 17* should be greater following the surgical procedure. In simple terms, the difference between DL and Dc can be explained by the fact that the lenticule 26 can be assumed to be assembled from two constituent parts 26a and 26b. Ultimately, the constituent part 26a corresponds to the change in curvature for the cornea 17* following the procedure. It thus has the height Dc in this simplified explanation. The remaining part 26b, into which the implant 26 can be thought to be subdivided, corresponds to the lenticule 21. This allows use to be made of a lenticule 21 which would not be suitable per se for the desired effect—in this case an increase in the curvature of the cornea 17. As a result of this matching, the lenticule 21 and the implant 26 have the same basic shape, specifically a greater thickness at the center than at the edge.

In this context, FIG. 6 shows a simplification. It is based on the fact that the difference between the bottom side of the lenticule/implant and the top side of the lenticule/implant is relevant to the change in the curvature of the cornea of the eye, with it coming down to the difference in the curvature of these two sides. By contrast, the absolute thickness of the lenticule/implant does not play a decisive role for the refractive change, which is to say the change in the curvature of the corneal front side. For example, this can be identified from the fact that the insertion or removal of a volume with a constant thickness over its lateral extent would change the curvature of the corneal front side only very slightly in the inserted region, and would instead cause the corneal front side to be located slightly further in the anterior or posterior direction, but approximately the same curvature would be maintained. FIG. 6 therefore shows the lenticule 21 very schematically with a greater edge thickness, since what this achieves is that the volume of the removed lenticule 21 is greater than that of the implant 26 to be inserted. The advantages of this non-mandatory option will be explained below. However, the schematic illustration in FIG. 6 must not be linked to the presumption that the edge thickness of the lenticule 21 would contribute to a change in the curvature of the corneal front side to an extent sufficient for the refraction alteration. This is not the case. Instead, with regards to the lenticule 21, it is predominantly the differences in curvature between the surfaces 22 and 23 that are relevant to the refraction correction, and these curvature differences are also plotted for the part 26b in the schematic illustration of the lenticule 26. This simplification therefore must not be mistaken to mean that the lenticule 21 is designed exactly in the shape of the part 26b with regards to the overall thickness. Although such a correspondence is possible, it is not mandatory.

The matching of lenticule 21 and implant 26 is achieved by virtue of the calculation device 16a initially defining the additive refraction alteration value for the implant 26 and subsequently adapting the lenticule 21 in respect of its subtractive refraction alteration value such that, overall, the desired change in curvature of the cornea 17* is obtained following the procedure. Here, the implant 26 in the exemplary embodiment of FIG. 6 has been chosen such that it implements an overcorrection. In other words, the implant 26 alone would bring about too great an increase in curvature on account of the additive refraction alteration value. In the illustrated example in the case of FIG. 6, this would have as a consequence that an existing hyperopia (cornea is too flat) is converted into a myopia (cornea is too steep). Nevertheless, this does not occur since the calculation device 16a follows up the corresponding choice of an overcorrecting implant 26 by setting the subtractive refraction alteration value for the lenticule 21, and hence for the cut surfaces 22, 23 which define the lenticule 21, such that, overall, precisely the desired correction is attained. Overall, this is possible as a result of combining two aspects. Firstly, the additive refractive error correction (inserting the implant 26) is combined with the subtractive refractive error correction (removing or taking away the lenticule 21). Moreover, this combination is implemented purposefully and starts with the definition of the implant 26 and matches the lenticule 21 thereto.

FIG. 7 shows a block diagram for a corresponding method for establishing the essential data. In a step S1, data about a refraction correction need of the eye are received; for example, these may have been provided in a preceding step possibly comprising a measurement of the eye in particular. The essential variable for an implant to be inserted is defined in a step S2 on the basis of this refraction correction need, and the implant is determined thereby. This variable is the additive refraction alteration value brought about by the implant.

Only the subtractive refraction alteration value, which is required on account of the refraction correction need and the additive refraction alteration brought about by the implant, is calculated in a step S3. As explained in the general part of the description and as will also be presented in detail below, steps S2 and S3 may be combined in this case. In a subsequent step S4, the lenticule to be isolated in the cornea is calculated on the basis of the subtractive refraction alteration value. In this case, the lenticule is designed such that its removal from the cornea brings about the required, complementary residual refraction correction. Hence, implant and lenticule in combination have as a consequence precisely the corneal modification required to cover the refraction correction need. The aforementioned volume matching can be brought about in the process.

The cut surface required to isolate the lenticule in the cornea is calculated in a final step S5. This cut surface (e.g., 22, 23 and an edge face for setting the overall thickness) which isolates the lenticule defines at the same time a chamber for the implant to be inserted. Appropriate data describing the cut surface are created for this cut surface. These data then serve as control data for correcting the refraction of the eye, wherein these may absolutely still be raw data which still have to be converted into appropriate actuation data for the treatment apparatus, for example by defining the aforementioned trajectories along which the focus is repositioned.

FIG. 8A shows a plan view of the cornea 17 of the eye for an embodiment in which an astigmatic correction should be undertaken. The sectional illustration of FIG. 8B plotted below shows the section in the horizontal axis through FIG. 8A. The lenticule 21 to be removed is plotted here. As is evident from the comparison with FIG. 8C, which depicts the sectional illustration through the vertical axis of FIG. 8A, the lenticule 21 is not rotationally symmetric as it has different curvatures in the two sections. Its basic structure is such that it would reduce the curvature of the corneal front side, like in FIG. 6 is well. Nevertheless, hyperopia correction is performed overall, with an increase in the curvature of the corneal front side. This is shown in FIG. 9, which shows a sectional illustration similar to that of FIG. 8A, albeit following the insertion of the implant 26, which is plotted with hatching in this case. The implant 26 has a basic shape similar to that of the removed lenticule which, as is very evident from FIG. 8C, is thicker in the middle than at the edge. Hence, the principle of FIG. 6 is also pursued here, albeit in the case of an astigmatic correction. In said FIG. 6, the implant 26 is rotationally symmetric, which is to say the illustration in FIG. 9 also applies equivalently (apart from the opening cut 24) to the horizontal sectional plane of FIG. 8A—at least with regards to the design of the implant 26 since the chamber created by the removal of the lenticule 21 is not rotationally symmetric.

FIG. 9 shows that problems do not arise in an edge zone 30, which is regularly problematic in the case of hyperopia corrections using the SMILE principle, since the basic shape of lenticule 21 and implant 6 is the same, with the result that no great stresses or steps arise in the edge zone 30, where the overlying membrane 25 comes together with the remainder of the cornea 17.

The use of a rotationally symmetric implant 26 has the further advantage that its rotational position, which is to say its rotary alignment about the dash-dotted axis in FIG. 9, is completely irrelevant when the implant 26 is inserted since the rotational asymmetry, which is to say the astigmatism, is achieved by the rotationally asymmetric chamber that is created by the removal of a rotationally asymmetric lenticule 21.

The procedure of starting the method with the determination of the implant in step S2 and of determining on that basis the refraction correction to be covered by the lenticule further has the advantage that work can be carried out with a set of implants in standard sizes. There is no need for an individual exact adaptation of the implant 26 to the specific refraction correction need, as this is achieved in step S3 by determining the subtractive refraction alteration value. An exemplary procedure for the use of such a set of standard implants 26 is explained below using the example of a hyperopia correction (S_{T}<0):
1. Inputting the manifest, which is to say presurgical, refractive error of an eye of a patient.
2. Inputting the target refraction (possibly also higher orders).
3. Calculating the sought-after refractive correction B_{T} as a difference; this is the refraction correction need. For example, if the postsurgical target refraction (residual refractive error) is 0 dpt and if the presurgical error is +5 dpt (i.e., hyperopia), a correction B_{T}=−5 dpt is sought-after. Thus, a hyperopia correction requires a correction by a negative diopter value, even if the presurgical visual error is described by a positive diopter specification.
4. Breaking the correction down into sphere, cylinder, higher orders (S_{T}, C_{T}, X_{T}).
5. Comparing the sphere S_{T} with a set Sᵢ: Selecting the additive correction strength by identifying the (positively or negatively) adjacent element from the set with Sᵢ>−S_{T} (in principle, each Sᵢ is suitable but the closest neighbor is preferred). If there is no departure from the example specified in 3., then the implant with Sᵢ=+6 dpt will be selected from a set of implants containing even diopter values since the following applies: B_{T}=−5 dpt=1 dpt−6 dpt. The Sᵢ are the (spherical) refractive power values of the implant 26. This determines the additive refraction alteration value. A myopia SMILE then removes a positively refractive lenticule 21 with the subtractive refraction alteration values B_{S} that are still missing. The calculation of the refractive power difference B_{S} to be obtained subtractively is implemented using the correction B_{T} and the chosen additive correction strength Sᵢ for providing the sought-after correction by a subtractive and an additive component: B_{T}=B_{S}−Sᵢ
6. Calculating the lenticular shape for correcting B_{S}. B_{S} describes the refractive power of the removed lenticule.
7. Creating data describing a cut surface which bounds the lenticule 21, and creating control data for creating the cut surface in the calculated form.

The implant 26 from the set which, in combination with the removal or taking of a lenticule suitably calculated for the refraction correction need, obtains the desired effect is selected for a patient, with the combination leading to a material-sparing operation.

In principle, the refractive power B_{S} of the lenticule removed can be positive or negative. This corresponds to a myopia correction (refractive power of the removed or taken lenticule is positive) in the first case and to a hyperopia correction per se in the second case, with the variant of the hyperopia correction being discussed in detail below. Higher orders in particular, but also spherical components, are possible, as are highly mixed variants. From an application stand-point, it is preferable in the case of hyperopia correction to completely place the subtractive component within the scope of SMILE myopia, which is to say ensure that the removal of the lenticule 21 brings about a negative refractive power correction along both principal meridians. In this way, the lenticule has a positive refractive power along both meridians and hence an advantageous physiological shape (middle thick, edge thin). However, this is not mandatory but merely an advantageous embodiment. In order for this to be achieved, the hyperopia is over-corrected by the implant 26 and a myopia correction is then performed subtractively in the cylinder component (as a minimum). This procedure, which appears odd at first glance, ensures that the implant concentrates on purely spherical hyperopia. This gives rise to the aforementioned advantage that there is no need to ensure a special orientation of the implant because the latter has no angle-dependent refractive power component. Then, the aforementioned method is modified as follows:
1. Inputting the manifest refractive error of an eye of a patient.
2. Inputting the target refraction (optionally also higher orders, multifocal property).
3. Calculating the sought-after refractive correction B_{T} as a difference (B_{T}=target=refraction).
4. Breaking the correction down into sphere S_{T_Max}, cylinder, higher orders (S_{T}, C_{T}, X_{T}).
5. Calculating the maximum B_{T_Max} and minimum refractive power B_{T_Max} in the corresponding meridians.
6. Comparing the sphere S_{T_Max} with the set Sᵢ: Selecting the additive correction strength by identifying the (positively or negatively) adjacent element from the set with Sᵢ>−S_{T_Max} (in principle, each Sᵢ is suitable but the closest neighbor is preferred). Calculating the meridional refractive power differences B_{S_Min} and B_{S_Min} to be obtained subtractively, from the correction B_{T} and the chosen additive corrective strength Sᵢ for the purpose of breaking the sought-after correction down into a subtractive and an additive component: B_{T_Max}=B_{S_Max}−Sᵢ and B_{T_Min}=B_{S_Min}−Sᵢ.
7. Calculating the lenticular form for correcting B_{S} (with B_{S_Max} and B_{S_Min}).
8. Creating control data for creating a lenticule of the calculated form.

In this case, higher-order corrections are preferably covered purely by the subtractive component while the correction of the respective adaptive sphere is implemented by the available implant 26 from the set. Thus, it is also an advantage of the combined additive-subtractive correction method that this reduces the number of implant variants required, reducing manufacturing and logistics outlay. There is no need for an implant 26 to be adjusted by the user.

A third advantage surprisingly arises to the effect that, within the scope of implanting into the corneal chamber that arises from the preceding lenticule extraction, the elasticity of the cornea 17 located thereabove as a cap 25 is now sufficient to completely adapt to the shape of the implant 26 since the dimensions of the lenticule 21 mean that significant lateral stretching is no longer required for the implant 26. In this respect, reference is made to the article by Gatinel et al. (Gatinel D., Weyhausen A., Bischoff M.; Journal of Refractive Surgery. 2020; 36 (12): 844-850). The subtractive method creates the space required for the implant 26. The formula for the simplified calculation of the volume of a SMILE treatment of myopia, specified by Gatinel et al., can be modified in order now to calculate the volume required for the implant 26. Equation 4 in the article is as follows:

$${V}_{L} \approx 0.5 {mm}^{3} - 0.28 \frac{{mm}^{3}}{dpt} S - 0.41 \frac{{mm}^{3}}{dpt} {C}^{-}$$

This equation applies in the negative cylinder notation and with a correction-related refractive power specification (change of sign) with the published constants for a minimum lenticular thickness (at the edge) of 15 μm, with which the absolute term scales. Moreover, the equation applies to diameters of the optical zone of 6.5 mm. Other constants are calculable accordingly by a person skilled in the art for other parameters.

In general, the following can be written for the subtractive volume (where S_{S} and C_{S} are the spherical and cylinder component, respectively, of the positive refractive power of the lenticule to be removed or taken away for the purpose of correcting the myopia):

$${V}_{S} \approx {a}_{S} + b \cdot {S}_{S} + c \cdot {{C}_{S}}^{-}$$

and the following can be written for the spherical additive volume:

$${V}_{A} \approx {a}_{A} + b \cdot {S}_{A}$$

Refractive powers of the lenticule and the implant apply as sign. Thus, the following inequality should hold with the requirement that the volume for the implant 26 should be created by the lenticule extraction and the diameters of the optical zones correspond:

V_{A}≤V_{S}

Alternatively, it is possible to formulate the requirement such that the inner arc lengths of the cap 25 (for all angles) are greater than or equal to the outer arc lengths of the implant 26, but the volume condition formulated here can serve as an approximation and is relatively easy to calculate. The following arises if the aforementioned equations are inserted into one another:

$${a}_{A} + b \cdot {S}_{A} \leq {a}_{S} + b \cdot {S}_{S} + c \cdot {{C}_{S}}^{-}$$

Consequently, the following condition arises for the additive sphere:

$${S}_{A} \leq \frac{{a}_{S} - {a}_{A}}{b} + {S}_{S} + \frac{c}{b} {{C}_{S}}^{-}$$

It is most likely that this condition can be satisfied if an edge thickness of the lenticule 21 that is as large as possible and an edge thickness of the implant 26 that is as small as possible cause a first term that is as large as possible. For example, it is possible to take a lenticule 21 with an edge thickness of 30 μm and implant an implant 26 with an edge thickness of 15 μm, whereby the denominator of the first term approximately becomes 1.0 mm³−0.5 mm³=0.5 mm³. A height difference of 15 μm arises at the edge in the process; this is feasible since this is the rule in the SMILE method in any case. The second term cannot be increased gainfully. Although increases in S_{S} are possible in theory, these would have to be compensated for by greater S_{A}. The third term is also predetermined. Thus, the following can also be written for these exemplary considerations (with the assumption that S_{S}=0):

$${S}_{A} \leq + 1.79 dpt + 1.46 {{C}_{S}}^{-}$$

Thus, a condition derived for the always negative variable S_{A} is that the volume of the implant 26 is no larger than the volume of the previously removed or taken lenticule 21 (if the diameter of the optical zone is the same). This is not shown by FIG. 6 on account of its schematic illustration.

If, as described herein, the implant is chosen such that this condition is observed, it may be the case that (deviating from the description above) it is not the implant 26 with the immediately adjacent refractive power value that is selected from the set. In that case, the implant 26 with which the aforementioned condition is approximately satisfied to the best extent is chosen.

For example, a hyperopic eye with S=+2.0 dpt and C=1.5 dpt is intended to be corrected completely (S_{T}=−2.0 dpt; C_{T}=−1.5 dpt). Thus, B_{T_Max}=0−(+2.0 dpt++1.5 dpt)=−3.5 dpt and B_{T_Min}=−20 dpt. From a set of spherical implants, the implant 26 with S₄=+4.0 dpt is selected (step S2 in FIG. 7).

S_{S}=B_{T_Max}−Sᵢ=−3.5 dpt++4.0 dpt=−0.5 dpt arises for the subtractive sphere and C_{S}=C_{T}=1.5 dpt for the cylinder component. Thus, on its own, the subtractively removed or taken lenticule 21 would perfectly correct myopia with S=−0.5 dpt and C=−1.5 dpt. Its calculation and creation in the eye of the patient are known to a person skilled in the art. Its surgical removal or taking away temporarily brings about an increase in the existing hyperopia to S=+4.0 dpt while at the same time completely correcting the cylinder (C=0 dpt). The purely spherical hyperopia remaining is now corrected by the incorporation of the selected implant 26. The application of the aforementioned inequality with C_{S}⁻=+1.5 dpt yields:

$${S}_{A} \leq + 3.98 dpt$$

This condition is approximately fulfilled by Sᵢ=+4.0 dpt. In principle, this is sufficient, wherein deviations of ±20%, preferably ±10%, particularly preferably ±5% may be admissible. The stress on the cap 25 caused by the implant 26 is very low in that case. Relief cuts can be dispensed with.

It is possible to accept optical zones of different sizes provided the smaller of the optical zones is large enough to cover the mesopic pupil. The correction range can be extended as a result.

All of these implementations are based on the fundamental concept of combining an additive and a subtractive correction. This embodiment variant on the basis of the volume comparison is an exemplary and preferred embodiment. The same type of considerations with regards to angle-dependent arc lengths can be taken into account analogously by a person skilled in the art. In particular, such an approach is more productive than the volume calculation with regards to the more accurate prediction of the residual astigmatism and corresponding countermeasures for minimizing same.

The implant may consist of donor tissue or an artificial tissue material.

Users themselves are able to produce an element of the set from a suitable blank. The manufacture in standard sizes from a blank is easier than the creation of a patient-individual special implant. This radically simplifies manufacturing and logistics, and the biomechanical problem becomes exactly solvable, at least in theory. To this end, the inlay produced by the user may also contain a cylinder component or other higher-order components. However, this means the implementation requires an axially correct implantation. Assistance methods in this respect (marks, shape adjustments, etc.) are known.

1.-22. (canceled)

23. A computer implemented method of creating control data for a treatment device for correcting refraction of an eye by way of corneal modification, comprising:
receiving data about a refraction correction need for the eye at an interface of the computer;
using a calculator operably connected to the interface of the computer to use the refraction correction need to determine an additive refraction alteration value resulting from an implant to be inserted into the cornea;
determining a subtractive refraction alteration value resulting from a lenticule to be removed from the cornea with the calculator;
the additive refraction alteration value and the subtractive refraction alteration value together yielding the refraction correction need;
using the subtractive refraction alteration value and the calculator to calculate the lenticule to be isolated in the cornea, in such a way that the lenticule is designed to bring about the subtractive refraction alteration value as a result of the lenticule's removal from the cornea; and
calculating a cut surface by application of the calculator such that the cut surface isolates the lenticule in the cornea and simultaneously bounds a chamber configured for the implant to be inserted therein and creating data describing the cut surface,
wherein:
the refractive correction need is a rotationally asymmetric correction, the additive refraction alteration value defines a purely rotationally symmetric refraction alteration, and the subtractive refraction alteration value defines a rotationally asymmetric refraction alteration.

24. The method as claimed in claim 23, further comprising determining the subtractive refraction alteration value from a difference between the refraction correction need and the additive refraction alteration value determined.

25. The method as claimed in claim 23, wherein the additive refraction alteration value defines a purely spherical refraction alteration, and the subtractive refraction alteration value defines an astigmatic refraction alteration.

26. The method as claimed in claim 23, further comprising determining the additive refraction alteration value to bring about an overcorrection of hyperopia or an overcorrection of myopia and determining the subtractive refraction alteration value to comprise a reversed hyperopia or myopia correction.

27. The method as claimed in claim 23, further comprising determining the additive refraction alteration value in such a way that a specified maximum deviation between additive refraction alteration value and refraction correction need is not exceeded.

28. The method as claimed in claim 23, further comprising, determining a presbyopia correction wherein the implant to be inserted is multifocal.

29. The method as claimed in in claim 23, further comprising, determining the lenticule and the implant to be thicker in a middle thereof than at an edge thereof, or determining the lenticule and the implant to be thinner at the middle thereof than at the edge thereof.

30. The method as claimed in in claim 24, further comprising providing a set of the implants, each of the implants of the set being configured for the additive refraction alteration resulting from insertion into the cornea, wherein the set comprises a plurality of implants each with an individual additive refraction alteration value and wherein the step of determining the implant comprises a selection of one of the implants.

31. The method as claimed in claim 30, further comprising selecting the implant from the set having the additive refraction alteration value closest to the refraction correction need on a basis of the refraction correction need.

32. The method as claimed in claim 23, further comprising determining a first volume of the lenticule to be greater than or equal to a second volume of the implant.

33. A method for correcting the refraction of an eye by way of corneal modification, comprising a method as claimed in claim 23 and further comprising:
using a laser apparatus to create the cut surface,
removing the lenticule from the cornea, and
inserting the implant into the chamber in the cornea that remains following the removal of the lenticule.

34. An apparatus for creating control data for a treatment device for correcting refraction of an eye by corneal modification, the apparatus comprising:
an interface that receives data about a refraction correction need for the eye;
a calculator connected to the interface and configured to receive the data regarding the refraction correction need;
to apply the refraction correction need to determine an additive refraction alteration value resulting from an implant to be inserted into the cornea;
to determine a subtractive refraction alteration value resulting from a lenticule to be removed from the cornea;
wherein the additive refraction alteration value and the subtractive refraction alteration value together yield the refraction correction need;
to use the subtractive refraction alteration value to calculate a lenticule to be isolated in the cornea, in such a way that the lenticule is configured to bring about the subtractive refraction alteration value as a result of removal of the lenticule from the cornea; and
to calculate a cut surface such that the cut surface isolates the lenticule in the cornea and simultaneously bounds a chamber configured to receive the implant to be inserted; and
to create data describing the cut surface,
wherein:
the correction is a rotationally asymmetric correction, the additive refraction alteration value defines a purely rotationally symmetric refraction alteration and the subtractive refraction alteration value defines a rotationally asymmetric refraction alteration.

35. The apparatus as claimed in claim 34, wherein the calculator is configured to determine the subtractive refraction alteration value from the difference between the refraction correction need and the additive refraction alteration value determined earlier.

36. The apparatus as claimed in claim 34, wherein the additive refraction alteration value defines a purely spherical refraction alteration, and the subtractive refraction alteration value defines an astigmatic refraction alteration.

37. The apparatus as claimed in claim 34, wherein the calculation device is configured to determine the additive refraction alteration value and the subtractive refraction alteration value in such a way that the additive refraction alteration value brings about an overcorrection of hyperopia or an overcorrection of myopia and the subtractive refraction alteration value comprises a reversed hyperopia correction or a reversed myopia correction.

38. The apparatus as claimed in claim 34, wherein the calculation device is configured to determine the additive refraction alteration value in such a way that a specified maximum deviation between additive refraction alteration value and refraction correction need is not exceeded.

39. The apparatus as claimed in claim 34, wherein the implant to be inserted is multifocal in order to bring about presbyopia correction.

40. The apparatus as claimed in claim 34, wherein the lenticule and the implant are thicker in a middle thereof than at an edge thereof, or the lenticule and the implant are thinner in the middle thereof than at the edge thereof.

41. A system comprising the apparatus as claimed in claim 34 and a set of implants, each of the implants being designed for the additive refraction alteration resulting from insertion of an implant into the cornea, wherein the set of implants comprises a plurality of implants each with an individual additive refraction alteration value and wherein the apparatus comprises an interface configured to receive data about the set of implants, wherein the data comprise the individual additive refraction alteration values and wherein the calculation device is configured to select one of the implants from the set when determining the additive refraction alteration value.

42. The system as claimed in claim 41, wherein the implants of the set each bring about a purely rotationally symmetric refraction alteration and the calculation device is configured to calculate the lenticule such that the lenticule brings about a rotationally asymmetric refraction alteration.

43. The system as claimed in claim 42, wherein the purely rotationally symmetric refraction alteration comprises a spherical refraction alteration and the rotationally asymmetric refraction alteration comprises an astigmatic refraction alteration.

44. The system as claimed in claim 43, wherein the calculation device is configured to select, on the basis of the refraction correction need, the implant from the set the refraction alteration value of which is closest to the refraction correction need.

45. A non-transitory computer program product that is not a carrier wave or signal having program code which, when loaded onto a computer, executes a method as claimed in claim 23.
